(19) United States
(12) Patent Application Publication
(10) Pub. No.: US 2022/0409376 A1
(43) Pub. Date: Dec. 29, 2022

(54) SYSTEMS, METHODS AND DEVICES FOR REPLACEMENT OF THE TEMPOROMANDIBULAR JOINT

(71) Applicant: MAXONIQ Pty Ltd, Carlton (AU)

(72) Inventor: George Dimitroulis, Carlton (AU)

(73) Assignee: MAXONIQ Pty Ltd, Carlton (AU)

(21) Appl. No.: 17/356,477

(22) Filed: Jun. 23, 2021

(51) Int. Cl. A61F 2/30

(52) U.S. Cl. CPC A61F 2/3099; A61F 2/30767; A61F 2002/30991; A61F 2002/30112; A61F 2310/00023

(57) ABSTRACT

A temporomandibular joint replacement system includes a condylar component secured with a mandibular bone and a fossa component with a fossa backing secured with a zygomatic bone, the fossa having an open-ended concave fossa dome with a post-center peak to allow for mediolateral and anterior translation with a rounded, oblong condylar head of the condylar component. The bone-interfacing surfaces are anatomically-contoured to their respective bone surfaces and formed with materials and textures which promote osseointegration with the joint replacement.

### BACKGROUND

### Field of the Invention

The embodiments described herein are related to temporomandibular joint replacements, and more particularly to a temporomandibular joint prosthesis with a fossa and joint shape which mimics a natural movement of the jaw.

### Related Art

The temporomandibular joint (TMJ) is a joint connecting the temporal bone of the skull to the mandibular bone of the jaw. The joint is complex, allowing for both anterior and posterior movement as well as mediolateral movement. This movement is generally accomplished by the interaction between a condylar head of the mandibular bone and a glenoid fossa of the temporal bone.

For a variety of reasons, the TMJ often wears down or fails—due to aging, accidents, disease or other physiological issues. In severe cases, it becomes necessary to replace the TMJ with a prosthesis which attempts to mimic the functionality and movement of the joint. This is exceedingly difficult due not only to the complexity of the joint itself, but to the desired movement in the mediolateral and anterior directions, along with the need for the joint to work with its corresponding joint on the opposite side of the jaw.

As a result of this complexity, TMJ replacements are often minimally effective at replacing and mimicking the TMJ. The replacement components which make up the condylar head and glenoid fossa often wear down, degrade or fail over time, requiring replacement of one or more of the prosthetic components. Replacement of a component which has been secured to the remaining bone structure is complex and often not possible. Additionally, the osseointegration of the existing prosthesis with the bone makes removal of failed prosthetic components even more difficult and unlikely.

Recent improvements in additive manufacturing have led to the development of customizable TMJ implants which have significantly improved upon the prior issues with regard to functionality, materials and movement. However, existing implants still struggle to provide sufficient structural support, accurately replicate the movement of the joint while also avoiding unnecessary wear and complications from failure of the implant to osseointegrate with the bone or allow tissue to reattach with bone structure around the implant. Failure of the interface between the condylar head and articulating surface of the fossa remain inevitable due the type of movement required and the amount of wear and tear experienced by the surfaces over time.

### SUMMARY

Embodiments of a temporomandibular joint replacement system described herein include a condylar component secured with a mandibular bone and a fossa component with a fossa backing secured with a zygomatic bone and fossa lining having an open-ended concave fossa dome with a post-center peak to allow for mediolateral and anterior translation with a rounded, oblong condylar head of the condylar component. The bone-interfacing surfaces are anatomically-contoured to their respective bone surfaces and formed with materials and textures which promote osseointegration with the joint replacement, while the fossa dome and condylar head are formed from materials suitable for durability and movement.

In one embodiment of the invention, a temporomandibular joint replacement comprises: a condylar component anatomically-contoured for attachment with a mandibular bone and having a condylar head; and a fossa component comprising: a fossa backing anatomically-contoured for attachment with a zygomatic bone; and a fossa lining including an open-ended concave articulating surface which interfaces with the condylar head.

In another embodiment of the invention, a temporomandibular joint replacement comprises a condylar component anatomically-contoured for attachment with a mandibular bone and having a condylar head, wherein the condylar head has a rounded, oblong shape; and a fossa component anatomically-contoured for attachment with a zygomatic bone and further comprising an open-ended concave articulating surface which interfaces with the condylar head, wherein the articulating surface of the fossa lining curves to a high point posterior of a center of a sagittal plane and apexes at a center of the fossa lining in a coronal plane.

In a further embodiment of the invention, a method of manufacturing a temporomandibular joint replacement comprises the steps of: forming an anatomically-contoured condyle component including a condylar head and a body portion for attachment with a mandibular bone; forming a fossa component including a fossa backing anatomically-contoured for attachment with a zygomatic bone and a fossa lining including an open-ended concave articulating surface which interfaces with the condylar head.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is a perspective-view illustration of a temporomandibular joint replacement (TMJ) prosthesis attached with a mandibular bone and a zygomatic bone of a patient, according to an embodiment of the invention;

FIG. 2 is a side-view illustration of the TMJ prosthesis including a fossa component and condyle component including a front-to-back curvature of a fossa dome interfacing with a condylar head, according to an embodiment of the invention;

FIG. 3 is a front transparent-view illustration of the TMJ prosthesis illustrating the interface between the condylar head and a side-to-side curvature of the fossa dome, according to an embodiment of the invention;

FIG. 4 is a detailed side-view illustration of the TMJ prosthesis illustrating the interface between the condylar head and fossa dome, according to one embodiment of the invention;

FIG. 5 is a front cross-sectional view illustration of the interface between the condylar head and fossa dome, according to one embodiment of the invention;

FIG. 6 is a bottom-view illustration of the interface of the condylar head with the fossa dome, according to an embodiment of the invention;

FIG. 7 is a side cross-sectional view illustration of the interface of the condylar head with the fossa dome, according to an embodiment of the invention;

FIG. 8 is a rear-view illustration of the fossa component, according to an embodiment of the invention;

FIG. 9 is an inferior-view illustration of the fossa component, according to an embodiment of the invention;

FIG. 10 is a side cross-sectional view illustration of the fossa component, according to an embodiment of the invention; and

FIG. 11 is a perspective view illustration of an outer surface of the condylar component, according to one embodiment of the invention;

FIG. 12 is a perspective view illustration of an anatomically-contoured interior surface of the condylar component, according to an embodiment of the invention; and

FIG. 13 is a side view illustration of the condylar head, according to an embodiment of the invention; and

FIG. 14 is a front view illustration of the condylar head, according to one embodiment of the invention;

FIG. 15 is a front cutaway view illustration of an articulating joint of the TMJ prosthesis, according to one embodiment of the invention;

FIG. 16 is a side-view illustration of the articulating joint of the temporomandibular joint replacement, according to one embodiment of the invention; and

FIG. 17 is a flow diagram illustrating an example process for manufacturing the TMJ prosthesis, according to an embodiment of the invention.

### DETAILED DESCRIPTION

Certain embodiments disclosed herein provide for a temporomandibular joint replacement system described herein include a condylar component secured with a mandibular bone and a fossa component with a fossa backing secured with a zygomatic bone, the fossa lining having an open-ended concave fossa dome with a post-center peak to allow for mediolateral and anterior translation with a rounded, oblong condylar head of the condylar component. The bone-interfacing surfaces are anatomically-contoured to their respective bone surfaces and formed with materials and textures which promote osseointegration with the joint replacement.

The temporomandibular joint (TMJ) replacement prosthesis replaces the mandibular condyle and augments the glenoid fossa to reconstruct a damaged or diseased TMJ when there is no alternative clinical avenue. The prosthesis is designed to mimic the natural range of motion of the TMJ by providing an articulating surface with a specified curvature that interfaces with an oblong condylar head to provide mediolateral and anterior motion similar to that of the natural joint.

This device is intended to be surgically implanted by suitably qualified oral and maxillofacial surgeons and/or plastic surgeons that have completed sufficient bridging training. The device may be used for patients who are typically suffering from intolerable symptoms of pain and/or jaw joint dysfunction who have failed to adequately respond to other treatments. This may include one or more of the following indications: osteoarthritis, other degenerative joint conditions, post-traumatic arthritis, rheumatoid arthritis, psoriatic arthritis, revision surgery, previously failed prosthetic joints, previously failed autogenous grafts, multiple operations on TMJ, developmental abnormalities, ankylosis, condylar hypoplasia, condylar resorption, benign tumors, osteochondroma and chondroma/osteoma.

The TMJ replacement prosthesis may be created using a three-dimensional medical image of a patient's bone structure in order to anatomically contour the implant to the individual patient. The implant may be created electronically via a software program which incorporates a three-dimensional (3D) image of the bone to create a corresponding 3D image of the implant that can then be further customized to create exact measurements of all aspects of the condylar components and fossa components that will fit the patient's anatomy. The implant components may then be additively-manufactured from a biocompatible material as a single piece to maximize the structural stability of the implant.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

### I. Temporomandibular Joint Replacement Prosthesis

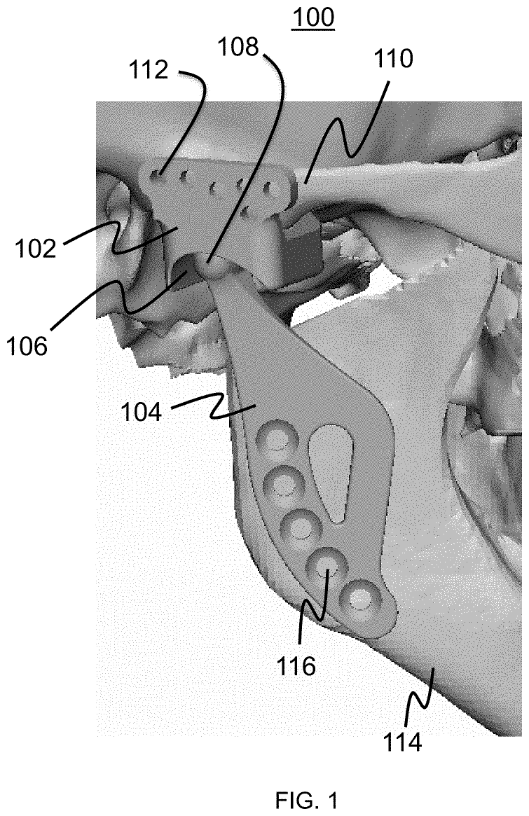

FIG. 1 is an illustration of one embodiment of a temporomandibular joint (TMJ) replacement prosthesis 100 after implantation. The TMJ replacement 100 primarily comprises a fossa component 102 and a condylar component 104 which are attached to respective portions of the bone structure of a human jaw and provide a replacement condyle and glenoid fossa to mimic the function of the TMJ. In the present embodiment, the fossa component 102 includes a fossa dome 106 with a concave articulating surface which interfaces with a condylar head 108 of the condylar component 104. As further illustrated in FIG. 1, the fossa component 102 is secured with a zygomatic arch of a zygomatic bone 110 via a plurality of bone screws inserted through screw openings 112 in the fossa component. Correspondingly, the condylar component 104 is secured with a ramus of a mandibular bone 114 via a plurality of bone screws inserted through screw openings 116 in the condylar component 104.

### II. Fossa Replacement Component

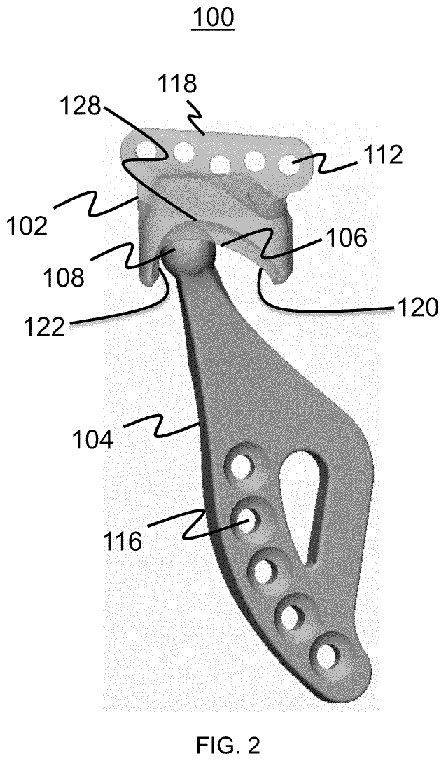

FIG. 2 is a detailed side-view illustration of TMJ prosthesis 100 including the fossa component 102 and condylar component 104 in an attached configuration. The fossa 102 includes a flange 118 which protrudes out from the main fossa body where the plurality of screw openings 112 are located. Also in FIG. 2, the side view shape of the articulating surface of the fossa dome 106 can be shown, which has a distinct articulation point 128 interfacing with a unique, oblong condylar head 108 to provide a similar shape and function as that of a natural TMJ. As shown, the fossa dome 106 provides an inner surface with a gradually curve from an anterior point 120 to its highest point (articulation point 128) posteriorly of a center in a sagittal plane. As the surface passes the articulation point 128, it has a steeper slope as it curves to a posterior point 122. This curvature holds the condylar head 108 in correct occlusion and allows for a more natural anterior and posterior movement.

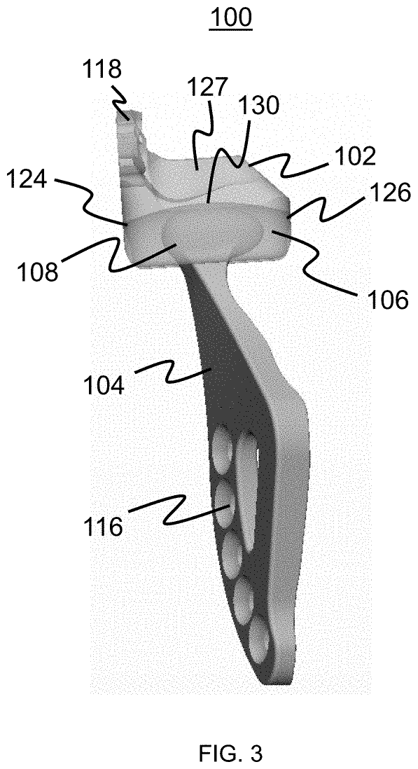

FIG. 3 is a front transparent-view illustration of the TMJ prosthesis 100 illustrating the interface between the condylar head 108 and a lateral curvature of the fossa dome 106 which has its apex 130 at the center of the fossa dome 106 in a coronal plane. In contrast with the anterior-posterior curvature of the fossa lining 106 seen in FIG. 2, the lateral curvature is more gradual and identical in slope from a left side 124 of the fossa dome 106 to a right side 126 of the fossa dome 106. This holds the condylar head 108 in correct occlusion to allow for a more natural mediolateral translation, which, in conjunction with the anterior-posterior curvature of the fossa dome 106, provides for movement of the entire joint that more accurately mimics the natural movement of the original TMJ.

As noted above, the fossa 102 includes a plurality of openings 112 in the flange 118 for inserting a plurality of bone screws to attach the fossa to the zygomatic arch. The number of openings 122 will vary depending on the particular shape and structure of each individual patient's anatomy but may vary from approximately four to approximately seven. Also shown in FIG. 3 is a bone-facing surface 127 of the fossa 102 which may be anatomically-shaped and contoured to fit the specific shape of a patient's bone structure to ensure an exact fit between the flange and the bone surface. In another embodiment, the bone-facing surface may be roughened to provide a textured surface to further encourage osseointegration of the fossa component with the bone structure.

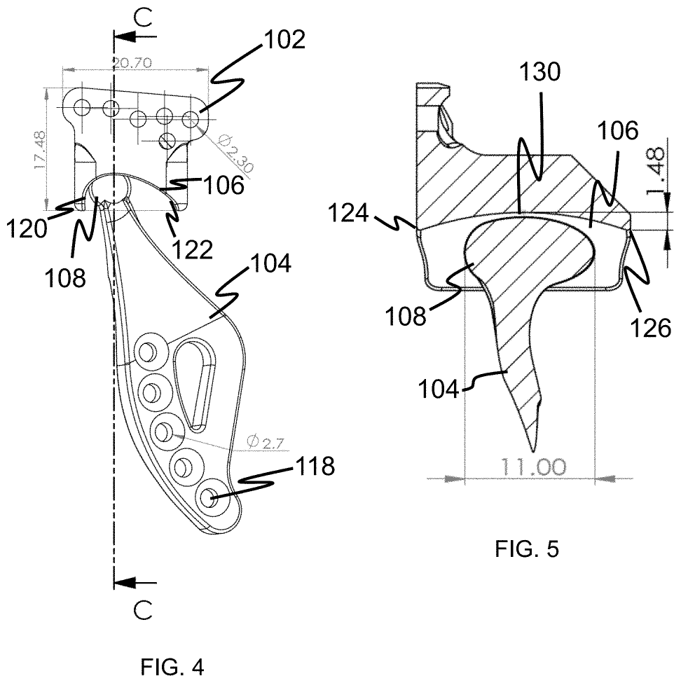

FIG. 4 is a detailed side-view illustration of the TMJ prosthesis illustrating the interface between the condylar head and fossa dome and showing a cross-sectional line “C” which is utilized for the front cross-sectional view illustration of the interface between the condylar head and fossa dome in FIG. 5. The gradual curvature of the fossa dome surface 106 is more clearly shown as it curves at the same slope from the left side 124 to the apex 130 at the center of the dome surface 106 and across to the right side 126.

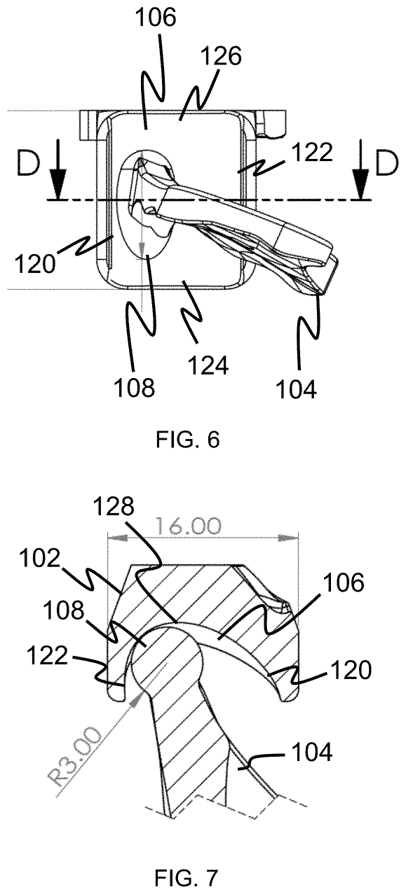

FIG. 6 is a bottom-view illustration of the interface of the condylar head 108 with the fossa dome 106, which shows the relationship of the oblong-shaped condylar head 108 the surface of the fossa dome. The fossa dome surface 106 extends the entire length of an inferior surface of the fossa 102, from the left side 124 to the right side 126, as well as from the anterior portion 120 to the posterior portion 122.

A lateral cross-sectional view illustration of the TMJ prosthesis is shown in FIG. 7 as taken from the line “D” in FIG. 6 extending from the anterior to the posterior of the fossa 102. This view more clearly illustrates the interface of the condylar head 108 with the unique anterior-posterior curvature of the fossa dome 106. As illustrated herein, the condylar head 108 is configured to primarily rest slightly posterior to the articulation point 128 that forms the apex of the anterior-posterior curvature of the fossa dome 106 extending from the anterior point 120 to the posterior point 122. The sharp curvature of the fossa dome 106 as it extends from the articulation point 128 to the posterior point 122 aids in keeping the condylar head 108 in a resting position when the joint is not being moved.

### III. Articulating Surface Geometry

The articulation point between the condylar head and fossa lining is designed to mimic the shape and function of the natural temporomandibular joint through the design of a uniquely-curved articulating surface on the fossa lining and an oblong curved shape of the condylar head, as will be described in further detail immediately below.

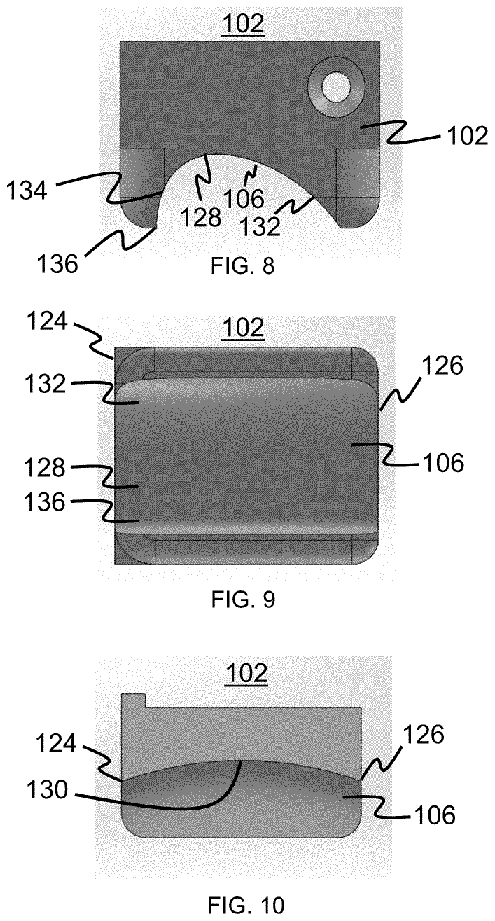

FIG. 8 is a rear-view illustration along a sagittal plane of one embodiment of the fossa 102, more clearly illustrating the geometry of the fossa dome articulating surface 106. The articulating surface 106 primarily has a gentle curved surface 132 which slowly curves to a high point 128 posterior to a center of the sagittal plane. After the high point 128, the curved surface continues at a significantly steeper slope 134 toward an inferior edge 136, which additionally helps to retain the condylar head at a position more suited for natural anterior movement of the joint, as illustrated above in FIG. 7.

FIG. 9 is an inferior-view illustration of the fossa surface 106 showing the overall length of the articulating surface 106 extending from the left portion 124 to the right portion 126. The gentle curved surface 106, apex 128 and steep curved surface 136 are identified for better understanding of the overall articulation points of the fossa lining.

FIG. 10 is a side cross-sectional view illustration of one embodiment of the fossa 192, illustrating the apex 130 at a center of the laterally-extending curved fossa dome 106 in a coronal plane as the curvature extends from the left edge 124 to the right edge 126.

### IV. Condyle & Condylar Head Geometry

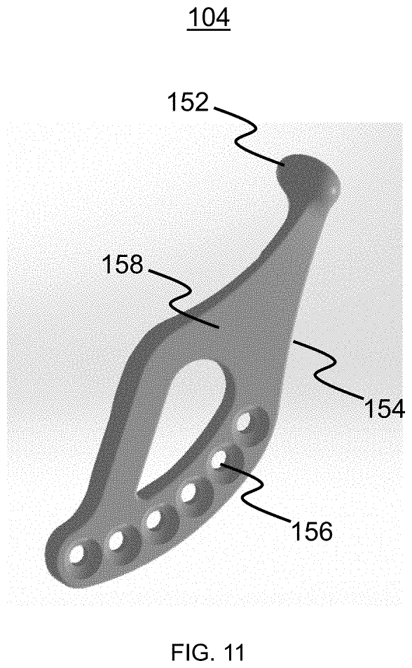

As illustrated in FIG. 11, in one embodiment the condyle component 104 has a rounded, oblong head 152 designed to fit into the articulating surface 124 of the fossa lining. The condylar component 104 extends away from the head 152 to form a body portion 154 which may be attached to a ramus 114 of the mandibular bone, as illustrated in FIG. 1. The body portion 154 may be secured to the ramus via a series of bone screws positioned through holes 156 in the body portion 154, with the locations being customized to the patient's anatomy. In one embodiment, approximately five to eight holes may be provided.

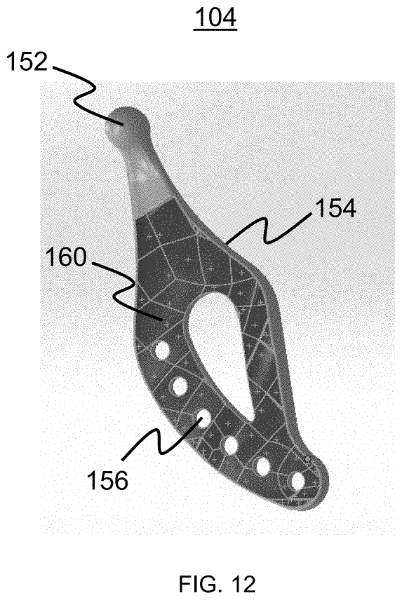

FIG. 11 illustrates an outer surface 158 of the condyle component 104, while FIG. 12 illustrates an interior surface 160 of the condyle component which faces the bone and is therefore contoured to fit an individual patient. Additionally, the interior surface 160 may also have a roughened surface to encourage osseointegration with the bone surface it is adjacent with.

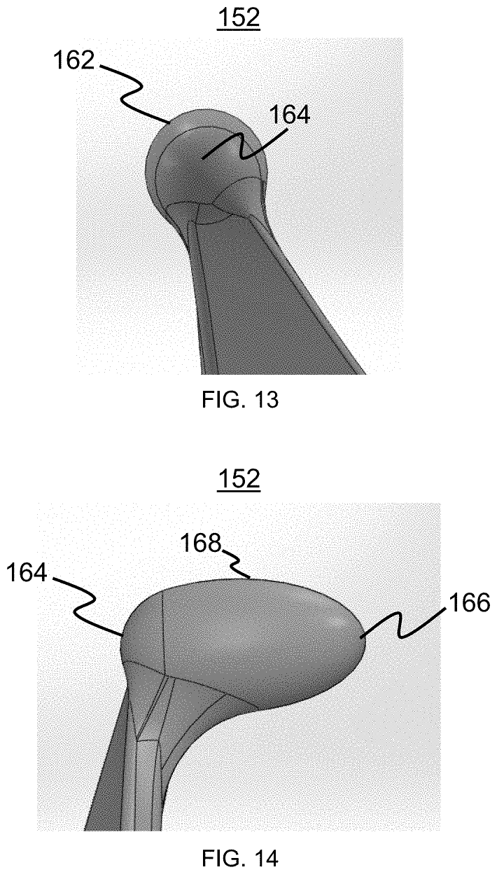

FIG. 13 is a side view illustration of a condylar head 152 taken along a sagittal plane, illustrating the overall spherical curvature 162 of the condylar head as viewed from the side perspective of directly viewing a left side 164 of the condylar head 152. In contrast, FIG. 14 is a front view illustration of the condylar head 152 viewed along a coronal plane, illustrating the oblong shape 168 of the condylar head as it extends from the left side 164 to a right side 166. The condylar head 152 is shaped to provide for rotation of the mandible for mastication and to also allow some mediolateral translation via the corresponding curvature and apex 150 of the articulating surface 124 of the fossa lining 106.

### V. Interface of Articulating Surface & Condylar Head

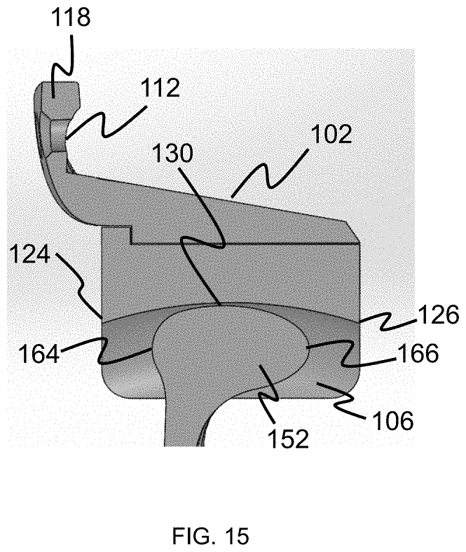

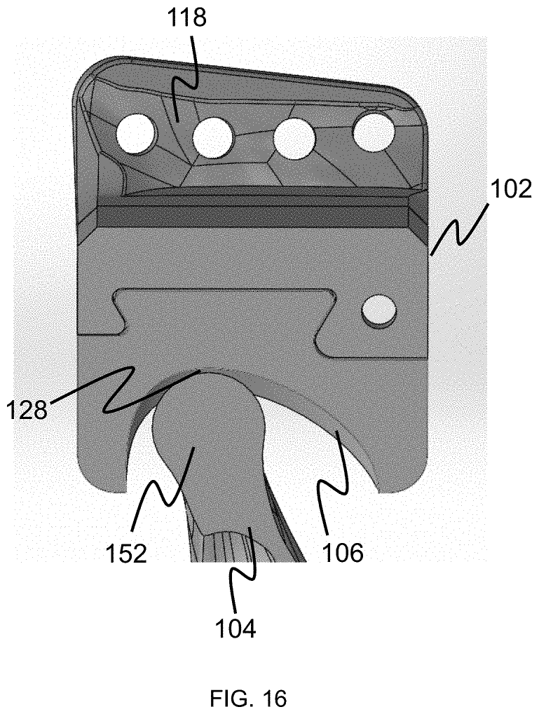

FIG. 15 is a front cross-sectional front-view illustration of one embodiment of an articulating joint of the temporomandibular joint replacement prosthesis 100, showing the placement of the condylar head 152 into the articulating surface 124 of the fossa 102. The condylar head 152 is positioned into the deepest part of the articulating surface 106 along the coronal plane, corresponding to the apex 130 previously illustrated in FIG. 10. FIG. 16 is a cross-sectional side-view illustration of one embodiment of the articulating joint prosthesis 100, further illustrating how the condylar head 152 is also positioned into the deepest part of the articulating surface 106 along the sagittal plane, corresponding to the high point 128 from FIG. 8.

Therefore, the shape of the articulating surface 106 and condylar head 152 maintain the natural occlusion of a patient's teeth, held in place by the patient's existing musculature, which allows for natural movement of the mandible both anteriorly and mediolaterally, restoring function to the jaw.

### VI. Materials

The components of the temporomandibular joint replacement prosthesis may vary depending upon the need for the materials to maintain structural integrity of the device, encourage osseointegration with adjacent bone structure, or resist repeated articulation movement. As noted above, the materials should be biocompatible and capable of use with additive manufacturing and milling so that each prosthesis can be anatomically-contoured and textured to each individual patient and corresponding bone structure.

In one embodiment, the fossa backing and condyle component may be manufactured with a titanium alloy such as Ti64 via three-dimensional (3D) printing and milling, with the bone-facing structures such as the interior flange portion 140, seat portion 146 and condyle interior surface 160 being manufactured and/or milled with a roughened surface to encourage osseointegration with the adjacent bone structure. The stabilization screw may also be manufactured with a titanium alloy.

The fossa lining may be manufactured from an ultra-high molecular weight polyethylene (UHMWPE) for use with the interface between the articulating surface of the fossa lining and the condylar head of the condylar component. The UHMWPE retains a smooth articulation surface during the interface with the condylar head.

### VII. Method of Manufacture

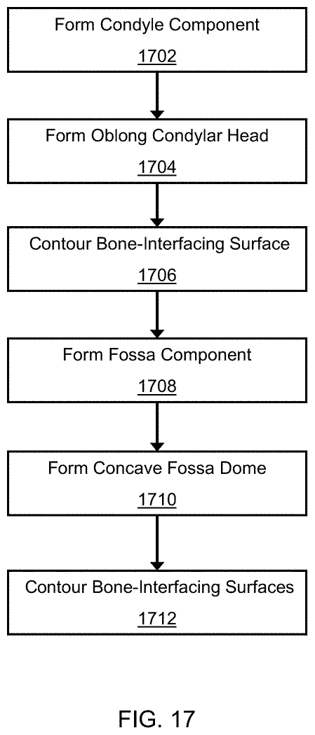

FIG. 17 is a flow diagram illustrating an example process for additively manufacturing the temporomandibular joint replacement prosthesis, according to an embodiment of the invention. In a first step 1702, the condylar component is formed in a shape which is anatomically-contoured to a bone structure of an individual patient. In step 1704, an oblong condylar head on the condylar component. In step 1706, an interior bone-interfacing surface of the condylar component is contoured to provide for enhanced osseointegration. In step 1708, a fossa component is formed which is anatomically-contoured to the bone structure of the patient. In step 1710, a concave fossa dome with a concave articulating surface is formed an interior surface of the fossa component. In step 1712, an interior bone-interfacing surface of the fossa component is contoured to provide for enhanced osseointegration.

As mentioned above, the temporomandibular jaw replacement prosthesis may be additively-manufactured from a biocompatible material so that each implant is anatomically-contoured to an individual patient. This may include the use of Ti64 and UHMWPE, as noted above.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

What is claimed is:

1. A temporomandibular joint replacement comprising:
a condylar component anatomically-contoured for attachment with a mandibular bone and having a condylar head; and
a fossa component comprising:
a fossa backing anatomically-contoured for attachment with a zygomatic bone; and
a fossa lining with an open-ended concave articulating surface which interfaces with the condylar head.

2. The temporomandibular joint replacement of claim 1, wherein the articulating surface of the fossa lining curves to a high point posterior of a center of a sagittal plane.

3. The temporomandibular joint replacement of claim 1, wherein the articulating surface of the fossa lining apexes at a center of the fossa lining in a coronal plane.

4. The temporomandibular joint replacement of claim 1, wherein the condylar head has a rounded, oblong shape.

5. The temporomandibular joint replacement of claim 4, wherein the oblong shape of the condylar head is oriented laterally within the articulating surface of the fossa lining.

6. The temporomandibular joint replacement of claim 1, wherein the articulating surface of the fossa lining is formed from an ultra-high molecular weight polyethylene.

7. The temporomandibular joint replacement of claim 1, wherein bone-interfacing surfaces of the fossa backing are formed from a titanium alloy.

8. The temporomandibular joint replacement of claim 1, wherein the bone-interfacing surfaces of the fossa backing have a roughened surface.

9. A method of manufacturing a temporomandibular joint replacement, comprising the steps of:
forming an anatomically-contoured condyle component including a condylar head and a body portion for attachment with a mandibular bone;
forming a fossa component including a fossa backing anatomically-contoured for attachment with a zygomatic bone and a fossa lining including an open-ended concave articulating surface which interfaces with the condylar head.

10. The method of claim 9, further comprising forming the articulating surface of the fossa lining such that it curves to a high point posterior of a center of a sagittal plane and apexes at a center of the fossa lining in a coronal plane.

11. The method of claim 9, further comprising forming the articulating surface of the fossa lining such that it apexes at a center of the fossa lining in a coronal plane.

12. The method of claim 9, further comprising forming the condylar head into a rounded, oblong shape.

13. The method of claim 9, further comprising forming the condyle and condylar head such that the oblong shape of the condylar head is oriented laterally within the articulating surface of the fossa lining.

14. The method of claim 9, further comprising forming the articulating surface of the fossa lining from an ultra-high molecular weight polyethylene.

15. The method of claim 9, further comprising forming the bone-interfacing surfaces of the fossa backing from a titanium alloy.

16. The method of claim 9, further comprising forming the bone-interfacing surfaces with a roughened surface.
